(19) United States
(12) Patent Application Publication
(10) Pub. No.: US 2006/0111696 A1
(43) Pub. Date: May 25, 2006

(54) Malleable ophthalmic surgery tubing

(76) Inventor: Michael D. Auld, Chesterfield, MO (US)

Correspondence Address: THOMPSON COBURN, LLP, ONE US BANK PLAZA, SUITE 3500, ST LOUIS, MO, 63101, US

(21) Appl. No.: 10/940,401

(22) Filed: Sep. 14, 2004

(51) Int. Cl. A61B 18/18

(52) U.S. Cl. 606/4; 606/16; 606/13

(57) ABSTRACT

A length of malleable tubing has opposite proximal and distal ends, with the proximal end being adapted for connection to a surgical light source or a surgical fluid source, and the distal end being adapted for attachment to an ophthalmic surgery instrument. The tubing has an interior bore that extends along the length of the tubing. A length of wire is secured to the tubing along the tubing length. The wire is malleable, and bending the wire holds the tubing in the bent configuration of the wire. In alternate embodiments of the tubing, the length of wire is secured to an interior surface of the tubing interior bore or is contained in the side wall of the tubing between the bore interior surface and the tubing exterior surface.

### BACKGROUND OF THE INVENTION

(1) Field of the Invention

The present invention pertains to a malleable length of tubing that is primarily intended for use with ophthalmic surgery instruments. More specifically, the present invention pertains to a length of tubing having opposite proximal and distal ends, where the proximal end is adapted for connection to a surgical light source or a surgical fluid source, and the distal end is adapted for attachment to an ophthalmic surgery instrument. The tubing has an interior bore that extends along the length of the tubing. A length of wire is secured to the tubing along the tubing length. The wire is malleable, and bending the wire holds the tubing in the bent configuration of the wire.

In alternate embodiments of the tubing, the length of wire is secured to an interior surface of the tubing interior bore, or is contained in the side wall of the tubing between the bore interior surface and the tubing exterior surface. Both embodiments of the invention may also include manually operable clips and/or moveable supports on the tubing.

(2) Description of the Prior Art

In many ophthalmic surgery procedures, it is necessary that a light source be provided to illuminate the interior of the eye, and that an additional surgical instrument be provided that can provide either aspiration, liquid infusion, or both to the interior of the eye. In addition to the above, it is also often necessary that a separate incision be provided in the eye that receives the ophthalmic surgery instrument being manipulated by the surgeon. Each of these instruments secured to the eye typically have a length of tubing that extends from the instrument. For example, the light provided to the interior of the eye is typically transmitted by a length of tubing containing one or more optic fibers. The tubing distal end is secured to the eye with the optic fiber distal end extending into the eye. The tubing proximal end is communicated with a light source. Aspiration and/or infusion of the eye interior is typically provided by a length of tubing secured to the eye at its distal end and communicating with a source of aspiration and/or infusion at its opposite proximal end. Often the surgical instrument being used by the surgeon also has a length of tubing extending from the instrument. For example, a microsurgical laser probe has a length of tubing containing the optic fiber length that conducts the laser light to the instrument.

It can be appreciated that the multiple lengths of tubing extending from the small area of the eye surgical site make it difficult for the surgeon to easily access the surgical site, and can add to the difficulty and mental strain of the surgeon in performing the surgery. Any modification to a microsurgical instrument that can organize the multiple lengths of tubing extending to the surgical site and simplify accessing the surgical site by the surgeon would provide a significant contribution to the field of ophthalmic surgery.

### SUMMARY OF THE INVENTION

The present invention seeks to control the positioning of one or more lengths of tubing extending to a ophthalmic surgery site, and thereby facilitate the surgeon's access to the surgical site. Basically, the present invention provides a length of malleable ophthalmic surgery tubing that may be used with a variety of different microsurgical instruments. For example, the malleable tubing of the invention may be used with a microsurgical laser probe, an illuminator, and an aspiration/infusion cannula, to name only a few examples of surgical instruments with which the tubing of the invention may be used.

The malleable ophthalmic surgery tubing is provided in any desired length that extends between opposite proximal and distal ends of the tubing. The distal end of the tubing is adapted for attachment to a surgical instrument such as the types described earlier. The proximal end of the tubing is adapted for attachment to a connector that connects the tubing proximal end to a source of illumination, aspiration and/or liquid infusion. The tubing has a hollow interior bore that extends along the length of the tubing. The interior bore is dimensioned to receive a length of optic fiber where the tubing is used with a source of illumination and a surgical instrument that provides light to the surgical site. In addition to the length of optic fiber, or in place of the length of optic fiber, the tubing interior bore also conducts aspiration and/or infused liquids between a source and the surgical site. The tubing is flexible along a majority of its length, and in particular the portion of the tubing length adjacent the surgical instrument. This enables the surgeon to easily manipulate the majority of the length of tubing.

The surgical tubing of the present invention differs from prior art tubing in that a length of wire is secured to the tubing along the tubing length. The wire is bendable into a bent configuration or shape, where the wire holds the tubing in the bent configuration or shape of the wire. This enables the portion of the tubing length adjacent the surgical site to be bent and moved to a position relative to the site where the length of the tubing does not interfere with easy access to the surgical site. In this manner, the malleable ophthalmic surgery tubing of the present invention facilitates the surgeon's access to the surgery site.

### BRIEF DESCRIPTION OF THE DRAWINGS

Further features of the malleable ophthalmic surgery tubing of the invention are set forth in the following detailed description of the invention and in the following drawing figures wherein:

FIG. 1 is a plan view of one example of an ophthalmic surgery instrument employing the malleable tubing of the invention;

FIG. 2 is a cross-section view through the malleable ophthalmic surgery tubing of FIG. 1;

FIG. 3 is an enlarged partial view of the distal end of the tubing of FIG. 1;

FIG. 4 is a plan view of a further embodiment of a surgical instrument employing the malleable ophthalmic surgery tubing of the invention;

FIG. 5 is an enlarged partial view of the distal end of the tubing of FIG. 4; and

FIG. 6 is cross-section of the malleable ophthalmic surgery tubing of FIG. 4.

### DETAILED DESCRIPTION OF THE PREFERRED EMBODIMENT OF THE INVENTION

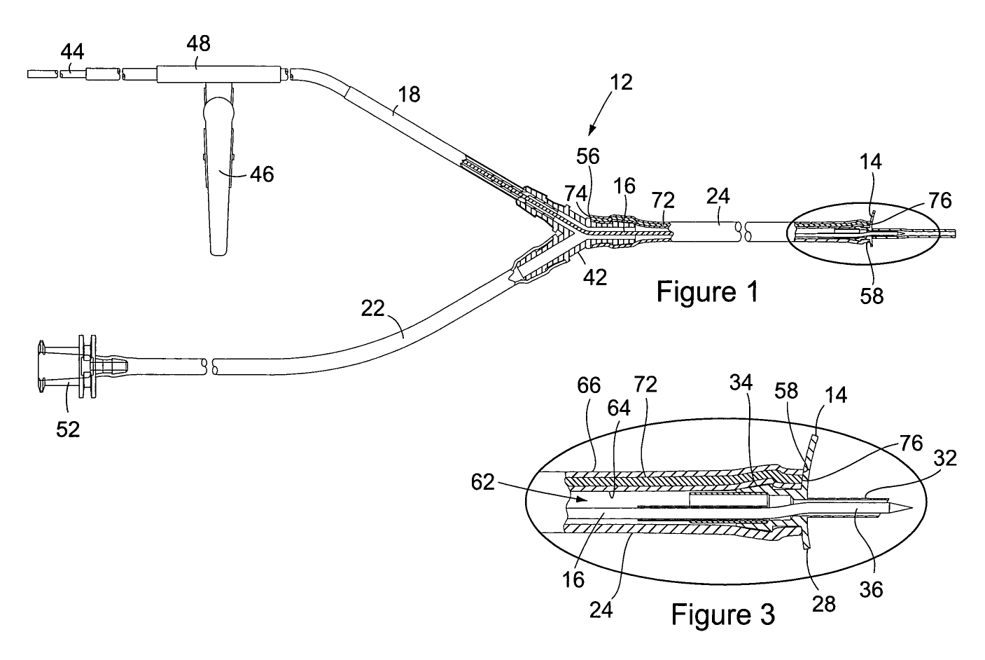

FIG. 1 shows one embodiment of a microsurgical instrument, an illuminator, employing the malleable ophthalmic surgery tubing of the invention. Although the apparatus and method of the invention are described with reference to a microsurgical illuminator 12, it should be understood that the apparatus and method of the invention are equally well suited for use with other types of surgical instruments that include a length of tubing having an interior bore that contains and protects a length of optic fiber extending through the bore, and/or having an interior bore that communicates the surgical instrument with a source of aspiration and/or infusion fluids, as well as other types of surgical instruments. Thus, the description of the malleable ophthalmic surgery tubing of the invention being used with a particular type of surgical instrument is intended to be illustrative only, and should not be interpreted as limiting the use of the tubing of the invention with any one particular type of instrument.

As shown in FIG. 1, the microsurgical instrument 12 is basically comprised of the instrument tip 14 that projects illumination, a length of optic fiber 16 that extends through the instrument 12, a length of fiber tubing 18 that surrounds and protects a portion of the optic fiber 16, a length of fluid transmitting tubing 22, and the malleable ophthalmic surgery tubing 24 of the present invention. Because the malleable tubing 24 of the invention may be employed with a variety of different types of surgical instruments as explained earlier, the component parts of the microsurgical illuminator 12 shown in FIGS. 1 and 3 will not be described in detail.

The illuminator tip 14 has a curved base plate 28 that is adapted to be secured to the surface of the eye at a desired surgical site. A tubular cannula 32 projects a short distance from the concave surface of the base plate 28. A hollow collar 34 projects from the opposite convex surface of the base plate 28. The interior bores of the cannula 32 and collar 34 receive a distal end portion 36 of the optic fiber 16.

The optic fiber 16 extends from its distal end portion 36 through the malleable ophthalmic tubing 24 of the invention, through a Y-connector 42, and through the optic tubing 18 to the proximal end portion of the optic fiber contained in a light source connector 44. The particular light source connector 44 shown in FIG. 1 is only one example of a light source connector that could be employed with the surgical instrument of the invention.

A manually operable clip 46 is attached to the optic tubing 18. The clip 46 includes a sleeve 48 mounted around the optic tubing 18. The sleeve 48 is dimensioned to slide over the exterior surface of the optic tubing 18. The clip 46 is operable to removably secure the optic tubing 18 and the surgical instrument 12 to a separate object to support the instrument. As is conventional, with the light source connector 44 connected to a source of light, the optic fiber 16 transmits the light through the surgical instrument 12 to the distal end portion 36 of the optic fiber where the light is projected on the surgical site.

The fluid tubing 22 extends from its distal end connected to the Y-connector 42 to its proximal end connected to a fluid source connector 52. The fluid source connecter 52 shown in FIG. 1 is only one example of a fluid source connector that could be used with the instrument 12 of the invention. As is conventional, the fluid source connector 52 is adapted to be connected to a source of fluid, either air pressure, suction, or liquid, and transmits the fluid through the length of the fluid tubing 22, through the interior of the Y-connector 42, and through the malleable tubing 24 and the instrument tip 14.

The malleable ophthalmic tubing 24 of the invention has a length with opposite proximal 56 and distal 58 ends. The overall length of the malleable tubing 24 can be chosen to best suit the instrument 12 for the particular surgical procedure for which it is to be used. The tubing 24 has an interior bore 62 that extends through the entire length of the tubing. The interior bore 62 is defined by a cylindrical interior surface 64 of the tubing. The tubing also has an opposite, generally cylindrical exterior surface 66. The tubing 24 is preferably constructed of a flexible material such as silicone. In the preferred embodiment the material is constant along the length of the tubing 24 and between the tubing interior surface 64 and exterior surface 66. In variant embodiments, other layers of materials may be added to reinforce the ophthalmic tubing or otherwise change the properties of the tubing. However, these variations of the tubing would not detract from the ability of the tubing to be bent and hold its bent configuration to be described.

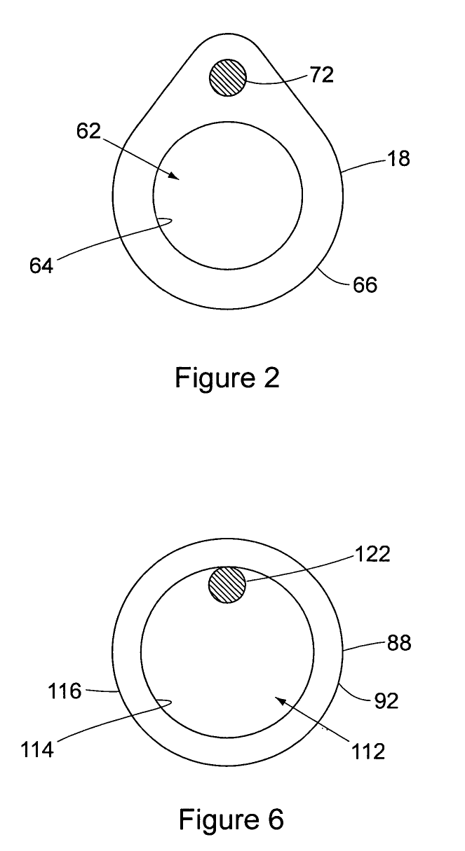

The malleable ophthalmic tubing 24 of the present invention differs from tubing of the prior art in that it is provided with a length of wire 72 that is secured to the tubing. The length of wire 72 extends along the tubing between a proximal end 74 of the wire at the tubing proximal end 56 and a distal end 76 of the wire at the tubing distal end 58. In the preferred embodiment, the wire 72 is a single stand of copper wire, although other types of materials may be employed. The single strand of wire 72 is preferred as it facilitates the ability of the surgeon to bend the wire in any desired configuration. In equivalent embodiments, multiple wires and braided wires could be used in lieu of the single wire 72. As seen in FIGS. 1, 2, and 3, the wire 72 is entirely contained in the side wall of the tubing 24 on one side of the tubing interior bore 62. As shown in FIG. 2, the wire 72 is encapsulated in the material of the tubing side wall between the tubing interior surface 64 and the tubing exterior surface 66. The dimensioning of the wire 72 and the material of the wire is chosen to enable the wire to be bent in any desired configuration and to remain in its bent configuration. In addition, the wire 72, when bent into its bent configuration must also hold the malleable ophthalmic tubing 24 in the bent configuration of the wire.

With the construction of the microsurgical instrument 12 described above, it can be seen that the malleable ophthalmic tubing 24 can be bent into any desired configuration by the surgeon and remain in that configuration. This enables the surgeon to configure and position the length of tubing 24 in any of a plurality of desired configurations and/or positions to provide unobstructed access to the surgical site.

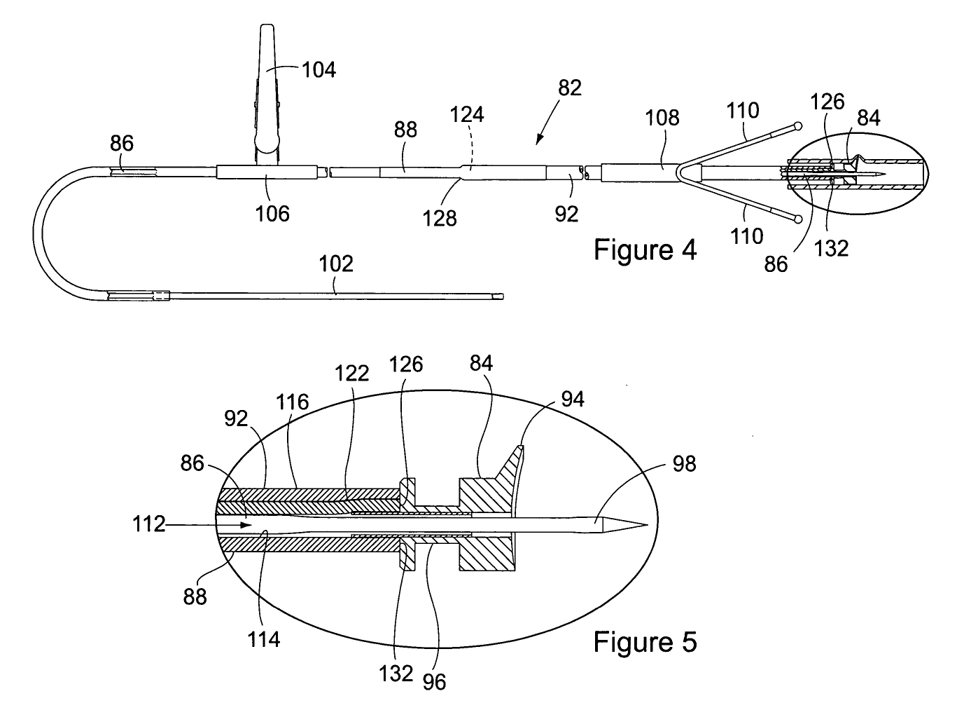

FIGS. 4, 5, and 6 show a variant embodiment of the microsurgical instrument 82. The surgical instrument 82 shown is another type of microsurgical illuminator. However, as explained earlier, the malleable ophthalmic tubing of the invention may be employed with other types of surgical instruments. Because the basic construction of the surgical instrument is known, it will not be described in detail. The surgical instrument 82 is basically comprised of an instrument tip 84, a length of optic fiber 86, and a length of surgical tubing 88, a portion of which is the malleable ophthalmic surgery tubing 92 of the invention.

The instrument tip 84, similar to the previously described embodiment, has a curved base plate 94 adapted for attachment to the surface of the eye at the desired surgical site. A cylindrical collar 96 projects from one side of the base plate 94. A hollow interior bore extends through both the collar 96 and the base plate 94 and receives a distal end portion 98 of the optic fiber 86.

The length of tubing 88 extends from the instrument tip collar 96 to a light source connector 102 at the opposite end of the tubing. As explained earlier, the light source connector 102 is only one type of connecter with which the surgical instrument 82 may be provided.

As in the previously described embodiment, the surgical instrument 82 is also provided with a manually operated clip 104 mounted by a sleeve 106 for movement along a portion of the length of the tubing 88.

Additionally, a V-shaped stabilizer 108 is mounted on the tubing 88 for sliding movement. The stabilizer 108 has a pair of bendable legs 110 that can be bent to conform to a surface to which the legs can be attached by surgical tape or other equivalent means.

As in the previously described embodiment, the length of tubing 88 has an interior bore 112 that is defined by a cylindrical interior surface 114 of the tubing. The bore 112 extends through the entire length of the tubing and provides a protective surrounding for the optic fiber 86. The side wall of the tubing is defined between the tubing cylindrical interior surface 114 and a cylindrical exterior surface 116 of the tubing.

The malleable portion of the tubing 96 is defined by a length of wire 122 contained in the portion of the tubing. The length of wire 122 is a single strand of wire that extends from a proximal end 124 of the wire to an opposite distal end 126 of the wire. The wire proximal end 74 defines the proximal end 128 of the malleable portion of the tubing and the wire distal end 76 defines the distal end 132 of the malleable portion of the tubing. This embodiment of the malleable tubing differs from the previously described embodiment in that an intermediate portion of the wire 72 is not secured to the tubing 92, but is free to move in the tubing interior bore 112. The entire length of the wire 122 extends through the tubing interior bore 112 and only the wire proximal end 124 and distal end 126 are secured to the tubing interior surface 114 by adhesives or other equivalent means. The length of wire 122 secured in the malleable portion of the tubing 92 enables the tubing portion to be bent in any desired configuration by the surgeon and positioned to provide the surgeon with unobstructed access to the surgical site.

While the present invention has been described by reference to specific embodiments, it should be understood that modifications and variations of the invention may be constructed without departing from the scope of the invention defined by the following claims.

What is claimed:

1. Surgical tubing comprising:
flexible tubing having a length with opposite proximal and distal ends, an interior bore extending through the tubing length, the tubing having an interior surface that surrounds the bore and an opposite exterior surface; and,
a wire secured to the tubing and extending along the tubing length, the wire being bendable into a bent configuration where the wire holds the tubing in the bent configuration of the wire.

2. The tubing of claim 1, further comprising:
a surgical instrument attached to the tubing distal end.

3. The tubing of claim 2, further comprising:
the surgical instrument having an interior bore that communicates with the tubing interior bore.

4. The tubing of claim 2, further comprising:
a light source connector communicating with the tubing proximal end and the tubing interior bore; and,
an optic fiber extending through the tubing, the optic fiber being bendable into a bent configuration where the wire holds the optic fiber in the bent configuration.

5. The tubing of claim 2, further comprising:
a fluid source connector communicating with the tubing proximal end and the tubing interior bore.

6. The tubing of claim 1, further comprising:
the wire extending through the tubing interior bore inside the tubing interior surface.

7. The tubing of claim 1, further comprising:
the wire extending through the tubing length inside the tubing exterior surface.

8. The tubing of claim 1, further comprising:
the wire extending through the tubing length between the tubing exterior surface and the tubing interior surface.

9. The tubing of claim 1, further comprising:
the tubing having a sidewall that surrounds the interior bore with the tubing interior surface and exterior surface being on the sidewall; and,
the wire extending along the tubing length inside the sidewall.

10. The tubing of claim 1, further comprising:
the wire having a length with an intermediate portion of the wire length extending between opposite proximal and distal ends of the wire, and the intermediate portion of the wire length being free to move in the tubing interior bore.

11. The tubing of claim 10, further comprising:
the wire distal end being secured to the tubing adjacent the tubing distal end.

12. The tubing of claim 10, further comprising:
the wire proximal end being secured to the tubing adjacent the tubing proximal end.

13. The tubing of claim 1, further comprising:
a clip attached to the tubing, the clip being manually operable to removably secure the tubing to a separate object.

14. The tubing of claim 1, further comprising:
a stabilizer attached to the tubing, the stabilizer having a pair of legs that project from the tubing and are securable to a separate object to support the tubing.

15. A method of positioning surgical tubing comprising:
providing a flexible length of tubing with opposite proximal and distal ends and an interior bore that extends through the tubing length; and,
securing a wire to the tubing with the wire extending along the tubing length, the wire being bendable and bending the wire into a bent configuration with the bent wire holding the tubing in the bent configuration of the wire.

16. The method of claim 15, further comprising:
attaching a surgical instrument at the tubing distal end; and,
positioning the surgical instrument at a surgical site with the bent wire directing the tubing in the bent configuration to the surgical instrument.

17. The method of claim 16, further comprising:
providing the surgical instrument with an interior bore and communicating the surgical instrument interior bore with the tubing interior bore.

18. The method of claim 16, further comprising:
attaching a light source connector at the tubing proximal end.

19. The method of claim 16, further comprising:
attaching a fluid source connector at the tubing proximal end.

20. The method of claim 15, further comprising:
providing an optic fiber extending through the tubing between the tubing proximal and distal ends.
